(19) United States
(12) Patent Application Publication
(10) Pub. No.: US 2006/0047341 A1
(43) Pub. Date: Mar. 2, 2006

(54) Spinal disc implants with reservoirs for delivery of therapeutic agents

(76) Inventor: Hai H. Trieu, Cordova, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 10/923,785

(22) Filed: Aug. 24, 2004

(51) Int. Cl. A61F 2/44

(52) U.S. Cl. 623/17.12; 623/17.16

(57) ABSTRACT

Nucleus pulposus implants that contain reservoirs for receiving, holding, and releasing therapeutic agents are provided. In one form of the invention, a spinal implant is provided with reservoirs positioned at least partially beneath the external surface of the implant. The reservoirs are provided to receive, hold, and release therapeutic and/or pharmaceutical agents into the surrounding tissues.

### FIELD OF THE INVENTION

The present invention relates to prosthetic spinal disc implants. More specifically, embodiments of the present invention relate to spinal disc implants with reservoirs for delivery of therapeutic and/or pharmaceutical agents to the surrounding tissues. Furthermore, the therapeutic agents and/or pharmaceutical agents can be replenished multiple times, before, during, or after surgical implantation.

### DESCRIPTION OF RELATED ART

The intervertebral disc functions to stabilize the spine and to distribute forces between vertebral bodies. A normal disc includes a gelatinous nucleus pulposus, an annulus fibrosis and two vertebral end plates. The nucleus pulposus is surrounded and confined by the annulus fibrosis.

Intervertebral discs may be displaced or damaged due to trauma or disease.

Disruption of the annulus fibrosis may allow the nucleus pulposus to protrude into the vertebral canal, a condition commonly referred to as a herniated or ruptured disc. The extruded nucleus pulposus may press on a spinal nerve, which may result in nerve damage, pain, numbness, muscle weakness and paralysis. Intervertebral discs also may deteriorate due to the normal aging process. As a disc dehydrates and hardens, the disc space height will be reduced, leading to instability of the spine, decreased mobility and pain.

One way to relieve the symptoms of these conditions is by surgical removal of a portion or the entire intervertebral disc. The removal of the damaged or unhealthy disc may allow the disc space to collapse, which would lead to instability of the spine, abnormal joint mechanics, nerve damage, as well as severe pain. Therefore, after removal of the disc, adjacent vertebrae are typically fused to preserve the disc space. Several devices exist to fill an intervertebral space following removal of all or part of the intervertebral disc in order to prevent disc space collapse and to promote fusion of adjacent vertebrae surrounding the disc space. Even though a certain degree of success with these devices has been achieved, full motion typically is never regained after such vertebral fusions. Attempts to overcome these problems have led to the development of disc replacement devices.

In addition to a replacement disc, or spinal implant, the prescribed treatment may also involve pharmacological agents to treat the diseased or damaged area, such as growth factors, antibiotics, and pain medication. The prescribed agents may include, for example, a growth factor to assist in repairing damaged endplates and/or the annulus fibrosis. Pharmacological agents also may be prescribed to prevent rejection of the implant, fight off infection, or provide pain relief for use after surgery. The agents may be prescribed separately or in combination.

U.S. Pat. No. 5,514,180 to Heggeness, et al. (“the '180 patent”), U.S. Pat. No. 6,033,438 to Bianchi, (“the '438 patent”), U.S. Pat. Nos. 6,146,420 and 5,702,449 to McKay, (“the '420 patent and the '449 patent,” respectively) and U.S. Pat. No. 6,620,196 to Trieu (“the '196 patent”) describe spinal implants that incorporate an osteogenic growth hormone to facilitate bone and/or tissue growth. However, these attempts do not allow for repeated refilling of these agents and do not describe the use of pain relievers, antibiotics, or other therapeutics and/or pharmaceuticals.

The '180 patent describes a mechanism by which an osteoinductive material may be incorporated into a prosthetic intervertebral device. More specifically, the material may be incorporated into some type of matrix, such as a collagen gel, prior to being formed or incorporated into the inventive intervertebral device.

The '438 patent describes an intervertebral spacer composed of bone. This device bears spinal loads and also provides a channel that can be packed with an osteogenic material. This material may include osteoinductive material to promote vertebral bone fusion to the device.

The '420 patent also describes an osteogenic fusion device. The device includes a collagen sheet soaked with a solution of a bone growth inducing substance such as a bone morphogenetic protein (BMP). The sheet then is wound around the central element of fusion device. The sheet is positioned so that it is in contact with the adjacent vertebral bone to promote fusion.

The '449 patent discloses a spinal implant which is comprised of a porous biocompatible material. The '449 patent further describes delivering a BMP to the site via the pores of the implant. Finally, the '196 patent discloses a hydrophilic implant that could advantageously deliver desired pharmacological agents. These agents could be BMP's, antibiotics, analgesics, or anti-inflammatory drugs.

These devices all function by delivering pharmacological agents into the prosthetic device to create bone fusion, but they are limited to inserting these agents prior to or during surgical implantation of the prosthetic. A need exists for a spinal implant that is capable of accepting therapeutic agents before, during, and/or after surgical implantation, holding those agents, and also providing in vivo delivery of those agents to the surrounding tissues. Furthermore, a need exists for a spinal implant that can be repeatedly replenished with therapeutic agents, and that can accept a wide range of therapeutic agents.

The description herein of problems and disadvantages of known apparatus, methods, and devices is not intended to limit the invention to the exclusion of these known entities. Indeed, embodiments of the invention may include one or more of the known apparatus, methods, and devices without suffering from the disadvantages and problems noted herein.

### SUMMARY OF THE INVENTION

A feature of an embodiment of the present invention provides a nucleus implant device that is capable of accepting therapeutic and/or pharmaceutical agents before, during, and/or after surgical implantation, holding those agents, and also providing in vivo delivery of these agents to the surrounding tissues. An additional feature of an embodiment of the invention provides a spinal implant that can be repeatedly replenished with therapeutic agents, and that can accept a wide range of therapeutic agents.

In accordance with these and other features of various embodiments of the invention, there is provided a spinal implant that contains reservoirs for receiving, holding, and releasing therapeutic and/or pharmaceutical agents. In one aspect of the present invention, spinal implants are provided that include a load bearing body sized for placement into an intervertebral disc space. Reservoirs are provided, preferably below an external surface of the implant, but the reservoirs remain in fluid communication with an external surface via channels or a series of pores, provided the spinal implant is fabricated from a relatively porous material.

In another embodiment of the present invention, the spinal implant described above is provided with multiple sets of reservoirs that will facilitate different release rates for the therapeutic agents contained therein. The multiple sets of reservoirs may or may not be in fluid communication with each other. The therapeutic agents that can be released to the surrounding tissues of the implant include pharmaceutical agents, biological agents, growth factors, analgesics, antibiotics, anti-inflammatory drugs, or any combination of drugs.

In accordance with another feature of an embodiment of the invention, there is provided a method of filling the implants. Therapeutic agents, preferably in liquid form, can be injected via a hypodermic needle (or other suitable delivery apparatus) into the reservoir. The reservoir may be filled with the desired quantity of therapeutic agents. Although it is particularly preferred that the needle be inserted through a predetermined injection site, the needle may be inserted anywhere on the implant, so long as the insertion does not adversely affect the life or function of the implant. While it is preferred that the therapeutic agents are in liquid form, it is also envisioned that the agents may be solid or substantially solid, and are delivered to the reservoirs via a powder or granule plunger, or other method known to those with ordinary skill in the art without undue experimentation.

In accordance with yet an additional feature of an embodiment of the invention, there is provided a method of fabricating a spinal implant containing at least one substantially solid therapeutic and/or pharmaceutical agents. In accordance with the method, therapeutic and/or pharmaceutical agents are provided in solid form and are suspended within a binding agent to create a pellet. In addition, another embodiment provides that the pellet of therapeutic agents is created from an extrusion of powder or granules of a therapeutic agent. A spinal implant then is formed or molded around the pellet. It is preferred that the pellet be of the same size and shape of the desired reservoir. After this implant is surgically implanted, water can diffuse through the implant and into the pellet, dissolving it. As the pellet dissolves, the therapeutic and/or pharmaceutical agents will be released to the surrounding tissues. After the pellet dissolves, a void will be left which is a reservoir that can be refilled using the method described above.

These and other objects and advantages of the present invention will be apparent from the description provide herein.

### BRIEF DESCRIPTION OF THE FIGURES

FIG. 1 illustrates a side view of a cross-section of a nucleus pulposus implant including reservoirs positioned in an intervertebral disc space.

FIG. 2 illustrates cross-sectional views of nucleus pulposus implants with reservoirs, varied in number, location, and geometry.

FIG. 3 illustrates cross-sectional views of a NAUTILUS™ nucleus pulposus implants with reservoirs, varied in number, location, and geometry.

FIG. 4 shows cross-sectional views of nucleus pulposus implants with reservoirs and channels.

FIG. 5 depicts cross-sectional views of nucleus pulposus implants with sets of reservoirs and varied amounts of channels.

FIG. 6 illustrates a preferred method for filling or refilling a nucleus pulposus implant with therapeutic agents in liquid form.

FIG. 7 illustrates a method for creating a nucleus pulposus implant around a substantially solid form of therapeutic and/or pharmaceutical agent.

### DESCRIPTION OF THE PREFERRED EMBODIMENTS

For the purposes of promoting an understanding of the present invention, reference will now be made to preferred embodiments and specific language will be used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a reference to “a spinal implant” includes a plurality of such implants, as well as a single implant, and a reference to “a therapeutic agent” is a reference to one or more therapeutic and/or pharmaceutical agents and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are cited for the purpose of describing and disclosing the various spinal implants, therapeutic and/or pharmaceutical agents, and other components that are reported in the publications and that might be used in connection with the invention. Nothing herein is to be construed as an admission that the invention is not entitled to antedate such disclosures by virtue of prior invention.

Throughout this description, the expression “substantially solid” as it refers to a substantially solid therapeutic and/or pharmaceutical agent that may be incorporated into a spinal implant, denotes an agent that is in tablet, pellet, capsule, powder, granule, flake, or gel form. Thus, the agent may not be completely solid, but may be surrounded by a solid capsule. In addition, the agent may be partially solid or gelatinous, and it is preferred that such partially solid materials substantially retain their shape during manufacture of the spinal implant. Throughout the description, the phrase “fluid communication” may mean diffusion, such as permeation, dialysis, osmosis, reverse osmosis, and ultrafiltration, all of which can occur through a membrane or another porous solid material; or may also mean internal flow through a pipe or duct, such as the channels that are incorporated in a preferred embodiment of the present invention.

In one aspect of the invention, an intervertebral spinal disc implant is configured to be a load bearing body of a size to be placed in an intervertebral disc space and intended to fully or partially replace the nucleus pulposus of mammals, particularly humans. In addition, these implants comprise at least one reservoir that is positioned at least partially inside the implant. The material of the implant preferably is either porous or incorporates channels to provide fluid communication between the reservoir and at least a portion of the external surface of the implant. The purpose of these reservoirs is to receive and hold therapeutic or pharmaceutical agents and provide in vivo release of these agents to the surrounding tissues. The therapeutic agents can be released into the body by diffusion. The therapeutic agents also can be released due to the cyclical loading that the implant is subjected to. As the implant is in the recipient's body, normal motions will place a cyclic loading on the implant. While not intending on being bound by any theory of operation, this cyclical compression is believed to increase the pressure within the implant and effectively pump the therapeutic agents out of the implant and into the surrounding tissues.

In preferred embodiments of the invention, the implant may include one or more reservoirs. These reservoirs may be in a variety of shapes and sizes, as well as orientations and locations within the implant. If there is more than one reservoir, the reservoirs may or may not be in fluid communication with each other. The implant also provides predetermined injection sites for repeated filling of these reservoirs, at any time, before, during, or after surgical implantation. In addition, these injection sites preferably are marked with a suitable marker (e.g., an x-ray marker) to assist in locating the injection sites under fluoroscopic guidance. It is also preferred that the implant have some form of self-sealing capabilities so that the injected therapeutic agents do not release out of the implant at a faster-than-desired rate. Therefore, a self-sealing valve is provided in one embodiment that will allow therapeutic agents to be injected but not leak out. Alternatively, the implant material itself will be self-sealing.

An additionally preferred embodiment of the invention includes a spinal implant that comprises multiple sets of reservoirs. These reservoirs preferably are contained within the implant body and are in fluid communication with at least a portion of the external surface of the implant body. The purpose for multiple sets of reservoirs is to allow multiple therapeutic and/or pharmaceutical agents to be released to the surrounding tissues, optionally with different rates of release.

Another embodiment of the invention pertains to methods of placing the therapeutic and/or pharmaceutical agents within the spinal implant. One method provides for injecting a solution of a therapeutic or pharmacological agent into the reservoir through one or more predetermined injection sites. These sites preferably are located by the use of a suitable marker (e.g., x-ray, etc.), thereby enabling the injection by fluoroscopic guidance. Another method provides for injecting a substantially solid form of a therapeutic or pharmacological agent into the reservoir using a suitable insertion apparatus. Yet another embodiment of the invention involves use of a therapeutic and/or pharmaceutical agent in substantially solid form. In this method, the spinal implant preferably is formed by molding or creating the implant around the substantially solid agent. When the implant is placed in the body, water may diffuse into the implant and into the pellet. The pellet then can dissolve, and therapeutic and/or pharmaceutical agents released into the surrounding tissues. Alternatively, water or other diluents can be administered to the substantially solid agent, either prior to or after insertion of the implant into the body, to cause the agent to dissolve.

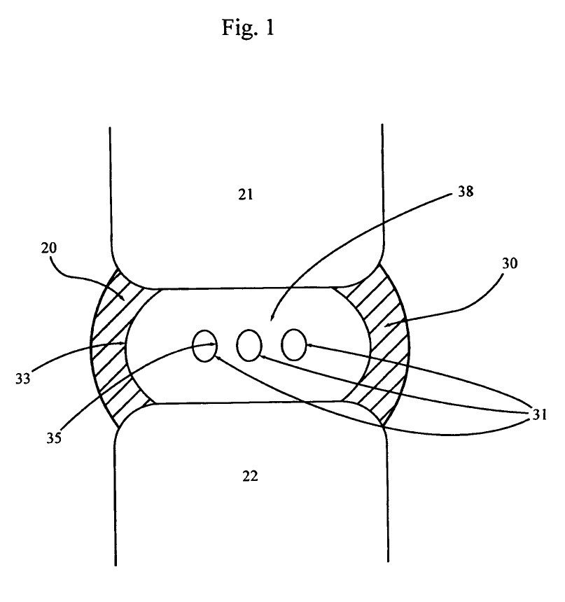

FIG. 1 illustrates a nucleus implant 30 implanted between a superior vertebral body 21 and an inferior vertebral body 22. The implant 30 preferably is at least partially surrounded by the annulus fibrosis 20. Implant 30 includes at least one reservoir 31, which preferably is an empty void within the implant material 38. Throughout this description, the term “reservoir” denotes an at least partially empty void, preferably an empty void, which may be filled with a solid or liquid therapeutic and/or pharmaceutical agent. The reservoir 31 preferably is formed entirely within at least one external surface of the implant 30, although reservoirs also exist when a portion of the reservoir is within at least one external surface of implant 30 (e.g., a depression on an external surface could be a reservoir in the context of the present invention). The reservoir 30 preferably is of a size large enough to contain an effective amount of a therapeutic agent.

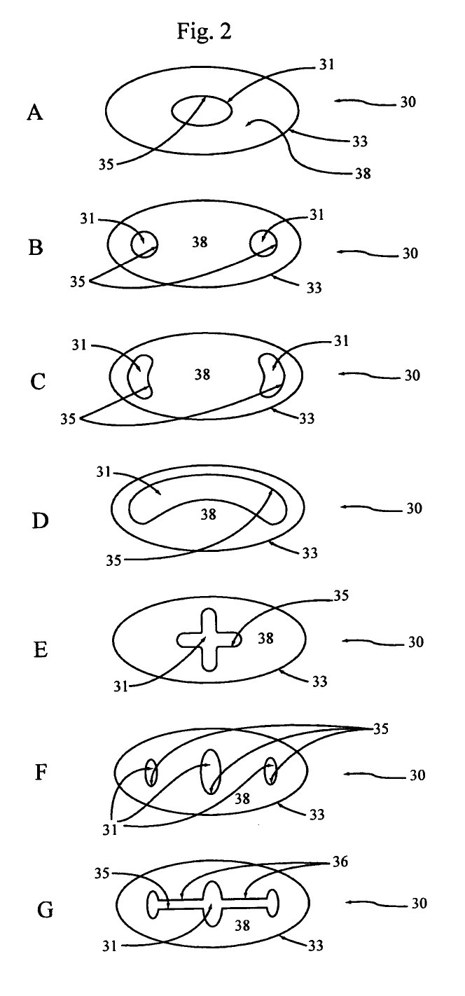

FIG. 2 illustrates various designs and possibilities for reservoir 31 positioned at least partially within an implant 30. The various configurations are designated as embodiments “A” through “G.” Embodiment A shows a centrally positioned reservoir 31, while B and C depict multiple reservoirs 31 that are positioned much closer to the implant's external surface 33. The shape of the reservoirs 31 in embodiments B and C are different: B encompassing a spherical shape, while C being kidney-shaped. Embodiment D depicts a single reservoir 31 that is formed and positioned in such a manner, that as much as the reservoir surface 35 area as possible can be in close proximity to the outer surface 33. The reservoir surface 35 is defined as the boundary between the void of the reservoir 31 and the material 38 used to fabricate the implant 30. The reservoir 31 also may be configured as shown in embodiment E to increase the reservoir surface 35 area for a set reservoir volume. This allows for the reservoir surface 35 to be in close proximity to the outer surface 33, which provides a lower fluid resistance between the reservoir 31 and the outer surface 33.

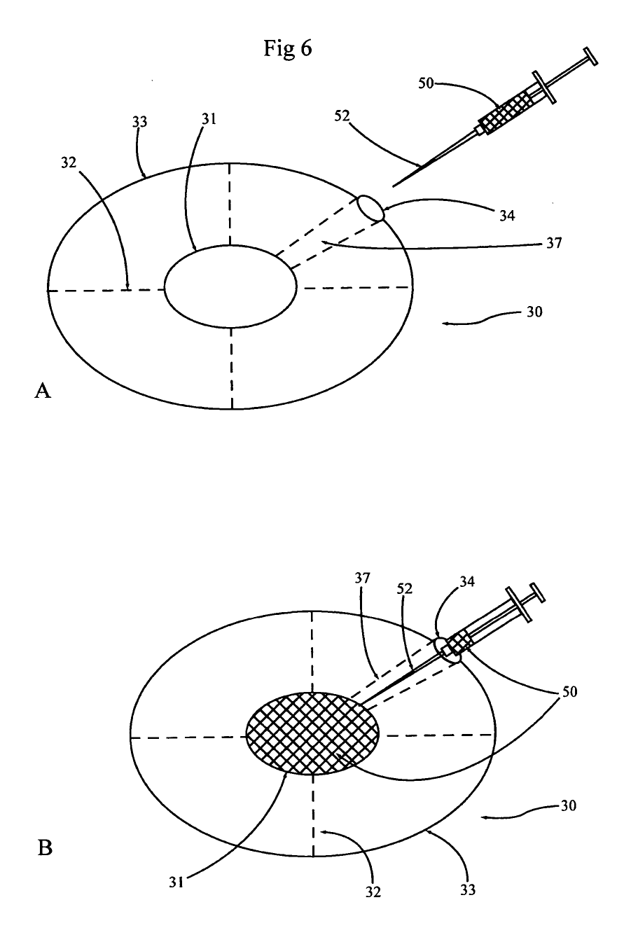

Embodiment F illustrates the implant 30 of the present invention with multiple reservoirs 31 dispersed throughout the implant 30. Embodiment G shows the same implant 30 with the same reservoirs 31 in fluid communication with each other via connecting channels 36. These connecting channels 36 preferably are comprised of voids in the implant material 38 that typically are made during manufacture of the implant 30. Multiple connected reservoirs 31 allow for all reservoirs to be filled through one predetermined injection site 34 (FIG. 6). This arrangement also ensures that no one reservoir 31 will drain of its therapeutic agents before the remainder of the reservoirs.

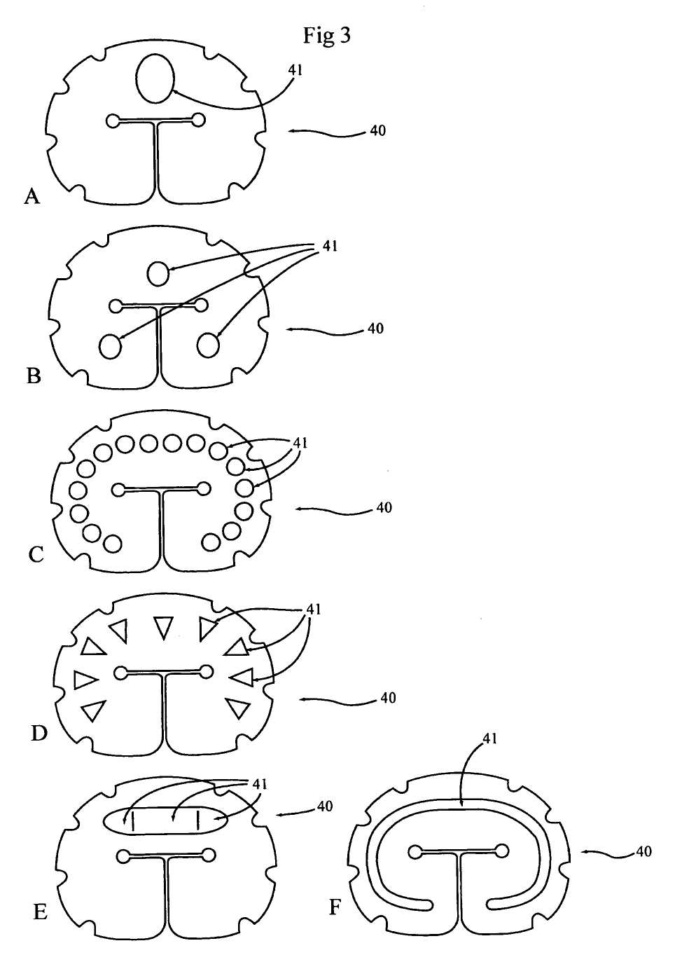

FIG. 3 illustrates various arrangements of reservoirs within a NAUTILUS shaped spinal implants 40, which are implants being developed by Medtronic Sofamor Danek, Memphis, Tenn. Again, the various embodiments depicted in FIG. 3 are denoted by reference letters as embodiments A-F. Embodiment A shows a single reservoir 41 that is centrally located, whereas embodiment B shows an implant 40 with multiple reservoirs 41 distributed throughout the implant 30. Embodiments C and D illustrate implants with a plurality of small reservoirs 41 positioned near an external surface of the implant 40. Embodiment E depicts a reservoir 41 with multiple chambers for holding multiple therapeutic and/or pharmaceutical agents. Finally, embodiment F shows one elongated reservoir 41 near the outer surface of implant 40.

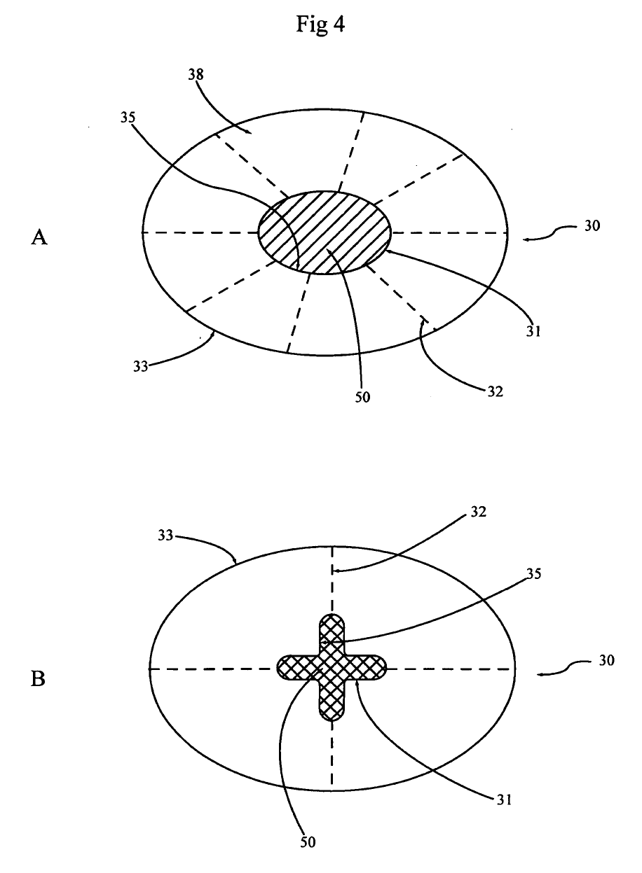

The present invention provides therapeutic and/or pharmaceutical agents to be delivered from the reservoir(s) 31, through the implant material 38, (FIG. 4) to the surrounding tissues. This can be accomplished by either making the implant material 38 from a relatively porous material, or by creating channels 32 throughout the implant material 38 as depicted in embodiments A and B of FIG. 4. To provide the therapeutic and/or pharmaceutical agent to the surface of the implant 30, it is preferred that there be fluid communication between the reservoirs 31 and at least a portion of the outer surface 33, regardless of how the fluid communication is accomplished. Channels 32 preferably are small tunnels or voids in the implant material 38 that extend through the reservoir surface 35 and the outer surface 33. Channels 32 can be made by forming the implant 30 around a small cylindrical wire or tube and then removing the wire or tube after formation to form a void. Alternatively, the material 38 used to form the implant may be relatively porous, such as a polymer matrix material that permits diffusion of fluids to and from the external surface 33 of the implant 38.

The implant material 38 can be comprised of a single material or it can be fabricated from multiple materials. The material or combination of materials chosen preferably will have load bearing properties to provide mechanical support to the spine as well as facilitate the in vivo release of the therapeutic agents 50. In addition, the material 38 should have a degree of flexibility to permit relative movement of the vertebral bodies between which the implant 30 is positioned. One possible material that can provide the mechanical support and release the therapeutic agents is a thermoplastic silicone polyurethane copolymer material.

While a silicone polyurethane polymer is a preferred material 38, implant 30 may be formed from a wide variety of biocompatible polymeric materials, including elastic materials, such as elastomeric materials, hydrogels or other hydrophilic polymers, or composites thereof. Suitable elastomers include silicone, polyurethane, copolymers of silicone and polyurethane, polyolefins, such as polyisobutylene rubber and polyisoprene rubber, neoprene rubber, nitrile rubber, vulcanized rubber and combinations thereof. The vulcanized rubber described herein may be produced, for example, by a vulcanization process utilizing a copolymer produced as described, for example, in U.S. Pat. No. 5,245,098 from 1-hexene and 5-methyl-1,4-hexadiene. Suitable hydrogels include natural hydrogels, and those formed from polyvinyl alcohol, acrylamides such as polyacrylic acid and poly(acrylonitrile-acrylic acid), polyurethanes, polyethylene glycol, poly(N-vinyl-2-pyrrolidone), acrylates such as poly(2-hydroxy ethyl methacrylate) and copolymers of acrylates with N-vinyl pyrrolidone, N-vinyl lactams, acrylamide, polyurethanes and polyacrylonitrile, or may be other similar materials that form a hydrogel. The hydrogel materials may further be cross-linked to provide further strength to the implant. Examples of polyurethanes include thermoplastic polyurethanes, aliphatic polyurethanes, segmented polyurethanes, hydrophilic polyurethanes, polyether-urethane, polycarbonate-urethane and silicone polyetherurethane. Other suitable hydrophilic polymers include naturally occurring materials such as glucomannan gel, hyaluronic acid, polysaccharides, such as cross-linked carboxyl-containing polysaccharides, and combinations thereof.

The implant 30 also may be comprised of a matrix or woven mass of any of the aforementioned polymers such that the implant 30 has a porosity sufficient to allow liquid therapeutic and/or pharmaceutical agents to diffuse to and from the external surface 33 of the implant 30. It is preferred that the porosity of the implant 30 in this preferred embodiment be at least above about 4%, more preferably above about 5%, and most preferably above about 10%. Using the guidelines provided herein, those skilled in the art will be capable of fabricating a suitable porous implant 30.

The nature of the materials employed to form the implant 30 should be selected so the formed implants have sufficient load bearing capacity. In preferred embodiments, a compressive strength of at least about 0.1 Mpa is desired, although compressive strengths in the range of about 1 Mpa to about 20 Mpa are more preferred.

The therapeutic agents 50, also referred to as pharmaceutical agents, biological agents, or growth factors, preferably are in a liquid form, e.g., in solution or slurry. Such agents may include, but are not limited to, antibiotics, analgesics, anesthetics, anti-inflammatory drugs, steroids, anti-viral and anti-retroviral compounds, therapeutic proteins or peptides, therapeutic nucleic acids (as naked plasmid or a component of an integrating or non-integrating gene therapy vector system), and combinations thereof.

Typical analgesics or anesthetics are non-steroidal anti-inflammatory drugs such as acetic acid derivatives, COX-2 selective inhibitors, COX-2 inhibitors, enolic acid derivatives, propionic acid derivatives, salicylic acid derivatives, opioids, opioid/nonopioid combination products, adjuvant analgesics, and general and regional/local anesthetics.

Antibiotics useful with the nucleus pulposus implants include, but are not limited to, amoxicillin, beta-lactamases, aminoglycosides, beta-lactam (glycopeptide), clindamycin, chloramphenicol, cephalosporins, ciprofloxacin, erythromycin, fluoroquinolones, macrolides, metronidazole, penicillins, quinolones, rapamycin, rifampin, streptomycin, sulfonamide, tetracyclines, trimethoprim, trimethoprim-sulfamthoxazole, and vancomycin. In addition, one skilled in the art of implant surgery or administrators of locations in which implant surgery occurs may prefer the introduction of one or more of the above-recited antibiotics to account for nosocomial infections or other factors specific to the location where the surgery is conducted. Accordingly, the invention further contemplates that one or more of the antibiotics recited supra, and any combination of one or more of the same antibiotics, may be included in the nucleus pulposus implants of the invention.

The invention further contemplates that immunosuppressives may be administered with the nucleus pulposus implants. Suitable immunosuppressive agents that may be administered in combination with the nucleus pulposus implants include, but are not limited to, steroids, cyclosporine, cyclosporine analogs, cyclophosphamide, methylprednisone, prednisone, azathioprine, FK-506, 15-deoxyspergualin, and other immunosuppressive agents that act by suppressing the function of responding T cells. Other immunosuppressive agents that may be administered in combination with the nucleus pulposus implants include, but are not limited to, prednisolone, methotrexate, thalidomide, methoxsalen, rapamycin, leflunomide, mizoribine (bredinin™), brequinar, deoxyspergualin, and azaspirane (SKF 105685), Orthoclone OKT™ 3 (muromonab-CD3). Sandimmune™, Neoral™, Sangdya™ (cyclosporine), Prograf™ (FK506, tacrolimus), Cellcept™ (mycophenolate motefil, of which the active metabolite is mycophenolic acid), Imuran™ (azathioprine), glucocorticosteroids, adrenocortical steroids such as Deltasone™ (prednisone) and Hydeltrasol™ (prednisolone), Folex™ and Mexate™ (methotrxate), Oxsoralen-Ultra™ (methoxsalen) and Rapamuen™ (sirolimus).

The invention also contemplates the use of therapeutic polynucleotides or polypeptides (hereinafter “growth factors”) with the nucleus pulposus implants of the invention. As noted supra, the growth factors are administered as proteins or peptides, or therapeutic nucleic acids, and may be administered as full-length proteins, mature forms thereof or domains thereof, as well as the polynucleotides encoding the same. Examples of therapeutic polypeptides include, but are not limited to, Bone Morphogenetic Proteins (BMPs), including BMP-1, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, and BMP-18; Vascular Endothelial Growth Factors (VEGFs), including VEGF-A, VEGF-B, VEGF-C, VEGF-D and VEGF-E; Connective Tissue Growth Factors (CTGFs), including CTGF-1, CTGF-2, and CTGF-3; Osteoprotegerin, Transforming Growth Factor betas (TGF-βs), including TGF-β-1, TGF-β-2, and TGF-β-3; and Platelet Derived Growth Factors (PDGFs), including PDGF-A, PDGF-B, PDGF-C, and PDGF-D. Other therapeutic polypeptides include inhibitors for tumor necrosis factors (e.g., anti-TNF α). The polynucleotides encoding the same may also be administered as gene therapy agents. In addition, the growth factors listed above may be used to advantageously repair the endplates, the annulus fibrosis, or any other tissues surrounding the implant.

BMPs are available from Genetics Institute, Inc., Cambridge, Mass. and also may be prepared by one skilled in the art, as described in U.S. Pat. No. 5,187,076 to Wozney et al.; U.S. Pat. No. 5,366,875 to Wozney et al.; U.S. Pat. No. 4,877,864 to Wang et al.; U.S. Pat. No. 5,108,922 to Wang et al.; U.S. Pat. No. 5,116,738 to Wang et al.; U.S. Pat. No. 5,013,649 to Wang et al.; U.S. Pat. No. 5,106,748 to Wozney et al.; and PCT Patent Nos. WO93/00432 to Wozney et al.; WO94/26893 to Celeste et al.; and WO94/26892 to Celeste et al. All bone morphogenic proteins are contemplated whether obtained as above or isolated from bone. Methods for isolating bone morphogenetic protein from bone are described, for example, in U.S. Pat. No. 4,294,753 to Urist and Urist et al., 81 PNAS 371, 1984.

In a particularly preferred embodiment of the invention, the nucleus pulposus implant comprises antagonists to either the myelin-associated glycoprotein (MAG) or Nogo-A, the largest transcript of the recently identified Nogo gene (formerly called NI-220), which are both present in CNS myelin and have been characterized as potent inhibitors of axonal growth. For example, Nogo-A acts as a potent neurite growth inhibitor in vitro and represses axonal regeneration and structural plasticity in the adult mammalian CNS in vivo. In another embodiment of the invention, antagonists to both MAG and Nogo-A are co-administered to the patient. In this preferred embodiment of the invention, the nucleus pulposus implants of the invention are used as implants for intervertebral discs that are adjacent locations of spinal cord injury, and may also replace damaged or infected endogenous nucleus pulposus. In this embodiment of the invention, the inhibitory activity of the antagonist(s) to the activity of MAG and Nogo-A may aid in the regeneration of damaged spinal nerve tissue, and the nucleus pulposus implant serves as a local reservoir of therapeutic antagonist(s) to aid in the growth of damaged spinal tissue. Antagonists of MAG and Nogo-A may take the form of monoclonal antibodies, anti-sense molecules, small molecule antagonists, and any other forms of protein antagonists known to those of skill in the art.

In this embodiment, therapeutic polypeptides or polynucleotides of Ninjurin-1 and Ninjurin-2 may further be administered alone or in conjunction with one or more MAG or Nogo-A antagonists, as a component of the nucleus pulposus implant. Ninjurin-1 and Ninjurin-2 are believed to promote neurite outgrowth from primary cultured dorsal root ganglion neurons. Ninjurin-1 is a gene that is up-regulated after nerve injury both in dorsal root ganglion (DRG) neurons and in Schwann cells. The full-length proteins, mature forms or domains of the full-length proteins thereof may be administered as growth factors, as well as the polynucleotides encoding the same.

The above listed agents may be used to treat various spinal conditions, including, but not limited to, degenerative disc disease, spinal arthritis, spinal infection, spinal tumors, osteoporosis, and combinations thereof. These agents also can be used in therapeutically effective amounts, such amounts may be determined by the skilled artisan depending on the type of treatment desired, the weight of the patient, the particular therapeutic agent, etc.

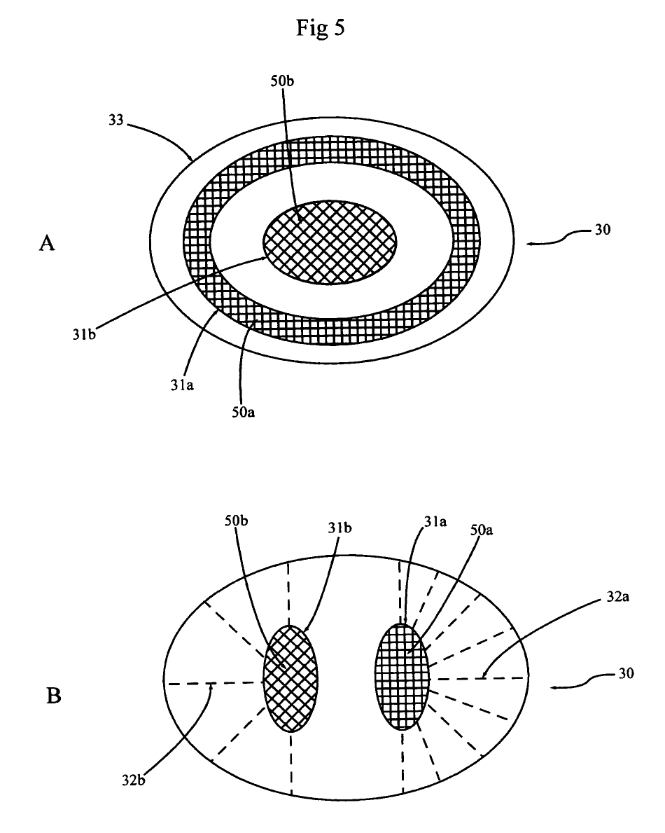

The attending physician may deem it necessary to prescribe multiple therapeutic agents 50 as the best therapy. Therefore, another embodiment of the present invention incorporates multiple sets of reservoirs 31 to accommodate multiple therapeutic agents 50. FIG. 5 (embodiments A and B) illustrates implants 30 with two reservoirs 31 (31a, 31b). Embodiment B depicts reservoir one 31a and reservoir two 31b. In this embodiment, the two reservoirs 31a, 31b are both about the same distance from the external surface 33, and typically would not be interconnected. Each reservoir 31a, 31b preferably has its own predetermined injection site 34 (not shown) and has its own set of channels 32a, 32b to provide delivery of the therapeutic agents 50.

At other times, the attending physician may find it necessary to prescribe multiple phases of pharmacological treatment, or may desire different release rates for the selected therapeutic agents 50. To achieve two separate rates of release, each reservoir 31a, 31b may be connected to different sets of channels 32a, 32b, with each set of channels being unique in number, or cross-sectional area. Reservoir one 31 a can be designated for phase I of treatment and reservoir two 31b designated for phase II of treatment. To adjust the rate of release, a reservoir can be connected to a larger or lower number of channels to decrease or increase the fluid resistance against the therapeutic agents, respectively.

Embodiment A illustrates another preferred feature of the present invention that creates varying rates of release of therapeutic agents 50. Reservoir one 31a is spanning the circumference of the implant 30, just below the outer surface 33, while reservoir two 31b is shown centrally located, much deeper within the implant 30. This arrangement is envisioned for an implant 30 that is either porous or incorporates channels 32 to provide fluid communication between the reservoirs 31a, 31b and at least a portion of the external surface 33. With reservoir one 31a nearer the surface 33, there is less fluid resistance from reservoir one 31a to the external surface 33 as compared to the fluid resistance between reservoir two 31b to the external surface 33. This arrangement will allow the first set of therapeutic agents 50a contained within reservoir one 31a to diffuse more quickly than the second set of therapeutic agents 50b contained within reservoir two 31b. This allows for multiple phases of treatment or gives the attending physician a choice of release rates for various therapeutic agents 50.

It also is envisioned that only one therapeutic agent be used in an implant with multiple reservoirs, however, the attending physician may want a choice in how quickly the therapeutic agents are released. Furthermore, adjusting the concentration, viscosity, or diffusivity of the solution or slurry of therapeutic agent 50 also can be used to adjust the release rate of the therapeutic agent 50. Furthermore, it is within the scope of the present invention to incorporate any number of reservoirs to establish different rates of release, or to position them in any orientation throughout the implant 30.

FIG. 6, embodiments A and B, illustrate a method for filling and refilling the reservoirs 31 with therapeutic and/or pharmaceutical agents 50. The implant 30 of the present invention preferably is provided with a predetermined injection site 34 that is in fluid communication with the reservoir 31 via an injection tube 37. The injection tube 37 may simply be a tube or void through the body of the implant 30, that connects the reservoir or reservoirs 31 to the predetermined injection site 37. The tube or body preferably is covered with a seal (not shown) that does not permit the release of agents from reservoir 31. The seal (not shown) may be self-sealing or a one-way valve, that allows the injection of the therapeutic agents 50, but does not allow those agents 50 to diffuse from the implant 30 at a higher-than-desired release rate.

A hypodermic needle 52 preferably is inserted into the predetermined injection site 34. A therapeutic agent 50 then is forced through the hypodermic needle 52 and into the reservoir 31. Ideally, the predetermined injection site 34 should be impervious to fluid, or at least have a higher fluid resistance than the channels 32, when not being used to fill the reservoir 31 (or it may contain a seal positioned on the exterior surface 33 of implant 30, much like a vial seal). As will be appreciated by those skilled in the art, if no seal is provided, and if an injection tube 37 is employed having a cross-sectional diameter much greater than the cross-sectional diameter of channels 32 (or greater than the effective pore size of porous implant 30 if a porous implant 30 is used), therapeutic and/or pharmaceutical agents 50 likely will leak back out of the predetermined injection site 34, and not through the channels 32. Thus, the desired rate of release may not be accomplished. A seal therefore is preferred in the invention. Alternatively, the injection tube may permit in vivo release of the agents, and is simply one or more of the channels 32 that are formed in implant 30 to enable dissipation of the therapeutic and/or pharmaceutical agent(s) 50. In addition, yet another embodiment provides that the implant material itself is self-sealing. In this embodiment, once the injection needle 52 is removed, the pressure that the implant is subjected to will compress and force close any opening created by the injection needle 52.

While it is preferred that the therapeutic agents are in liquid form, it also is envisioned that the agents may be delivered to the reservoirs via a powder or granule plunger, or other methods known to those having ordinary skill in the art. Once the implant 30 has been implanted in the body, water preferably will diffuse into the implant 30, through channels 32 or porous material 38. The agent pellet 51 will absorb water and dissolve. As the implant 30 is subjected to cyclical loading, the therapeutic agents 50 may diffuse and release into the surrounding tissues.

To avoid damage to the implant 30, it is preferred that the injection tube 37 be resilient to punctures. This will allow the needle 52 that enters the injection site to be guided straight to the reservoirs and not damage the implant 30 so as to shorten its useful life. Alternatively, the injection tube 37 could be large enough so that the hypodermic needle 52 only needs to be inserted to just below the surface 33, thus decreasing the risk of any errant puncture by the needle 52. In this arrangement, the injection tube 37 will need to be large enough so that the fluid resistance is low and can accommodate therapeutic agents 50 of varied viscosities to flow freely into the reservoir 31.

In yet another embodiment, it is envisioned to simply inject the therapeutic agent 50 through a needle 52 that is smaller in diameter than the channels 32. This will create a hole in the implant 30, but if it is small enough, it is not likely to greatly affect the release rate of the therapeutic agents 50. In addition, implant 30 may be formed of a material resilient enough to re-seal after puncture from a needle 52, thereby enabling direct injection into reservoirs 31 without the need for injection tube 37.

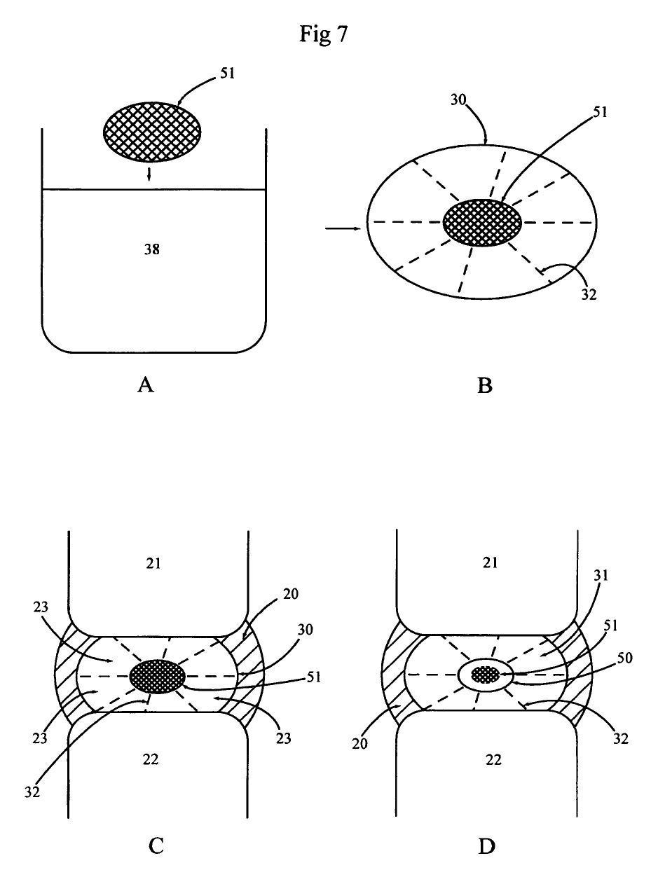

Another embodiment of the present invention involves forming the spinal implant 30 around a substantially solid therapeutic agent, as illustrated in FIG. 7, A-D. The therapeutic agents 50, in substantially solid form, preferably are dispersed in a binding agent to create an agent pellet 51. This agent pellet 51 preferably is a solid pellet, yet water soluble, although it may be in the form of a capsule, gel cap, gelatinous mass, and the like. The binding agent could be any binding agent known in the art useful in forming a tablet, such as hydroxypropyl methyl cellulose (HPMC), or hydroxymethyl cellulose (HMC), and other known binding agents.

The agent pellet 51 (or gel cap or capsule, etc.) preferably is formed in a shape that will essentially match the desired geometry of the reservoir 31, as it will be positioned at least partially within the implant 30 in the desired position and orientation. As shown at A of FIG. 7, the agent pellet 51 then may be placed in an uncured implant material 38 that will be used for molding or forming the spinal implant 30. The material 38 used to form the implant could be a thermoplastic polymer such as silicone polyurethane, or it could be any other material or a combination of materials. It is envisioned that the materials chosen will provide an optimal balance of sustaining the mechanical loads and stresses placed on a spinal implant as well as provide a suitable means of diffusion for the therapeutic agents 50 contained within.

Implant 30 then preferably is formed using conventional forming techniques, such as injection molding, thermoforming, extrusion, and other techniques known to those skilled in the art. The substantially solid agent pellet 51 can be placed in the molten polymer slurry or solution prior to entering the forming procedure, or after the polymer has begun to solidify during formation of the implant 30. This will permit the manufacturer to place the substantially solid agent pellet 51 in a desired location within the implant 30. An alternative embodiment envisions fabricating an outer shell of implant 30 first, allowing the implant material 30 to solidify, then placing the substantially solid agent pellet 51 within the shell and filling the remainder of the mold cavity with additional implant material 30. After solidification, the final implant 30 will include a substantially solid agent pellet 51 at least partially within its external surface.

Using the techniques described above, channels 32 also can be formed in the implant material 30 to permit diffusion of agent from substantially solid agent pellet 51, after the pellet begins to dissolve or disintegrate. For example, after implantation of implant 30, water, diluent or other liquid material can be administered to substantially solid agent pellet 51, or body fluids can diffuse inward through implant 30 (if porous) or through channels 32, to contact substantially solid agent pellet 51 and cause it to begin to dissolve or disintegrate. Once the substantially solid agent pellet 51 begins to dissolve, it will diffuse into the body, and leave an empty void. The empty void forms reservoir 31 that can then be refilled as described above. If the implant material 38 chosen is not porous, or not porous enough to facilitate diffusion, channels 32 can be formed in the implant 30. This can be done by cutting into the implant 30 with cutting tools such as needles or laser drilling, or the channels 32 can be formed during formation of implant 30 by placing channel formers (e.g., thin rods or wires) in the mold cavity. In addition, an injection tube 37 also can be created in the implant 30 for repeatedly refilling the reservoir 31.

Once the implant 30 has been implanted in the body, water 23 or body fluids preferably diffuse into the implant 30, through channels 32 or porous material 38. The agent pellet 51 will absorb water 23 or body fluids and dissolve or disintegrate. As the implant 30 is subjected to cyclical loading, the therapeutic agents 50 will diffuse and release into the surrounding tissues. Alternatively, the therapeutic agents 50 may diffuse and release into the surrounding tissue by other means, such as concentration gradient diffusion, osmosis, and the like.

The foregoing detailed description is provided to describe the invention in detail, and is not intended to limit the invention. Those skilled in the art will appreciate that various modifications may be made to the invention without departing significantly from the spirit and scope thereof.

What is claimed is:

1. A spinal implant, comprising:
a load bearing body, having an outer surface, sized for placement at least partially into an intervertebral disc space; and
at least one reservoir positioned at least partially within the body for holding and releasing therapeutic agents;
whereby the at least one reservoir is in fluid communication with at least a portion of the outer surface of the body, thereby providing for in vivo release of the therapeutic agents.

2. The implant of claim 1, wherein the body has one or more predetermined injection sites for depositing the therapeutic agents.

3. The implant of claim 2, wherein the one or more predetermined injection sites include a sealing mechanism to prevent release of the therapeutic agents into the surrounding tissues.

4. The implant of claim 2, wherein the one or more predetermined injection sites are marked with at least one x-ray marker.

5. The implant of claim 1, the implant further comprising one or more channels between the at least one reservoir and the outer surface of the body to permit fluid communication therebetween.

6. The implant of claim 1, wherein the at least one reservoir comprises more than one reservoir.

7. The implant of claim 6, wherein the more than one reservoir are not in fluid communication with each other.

8. The implant of claim 1, wherein the body is comprised of one or more materials providing load bearing properties and release of therapeutic agents.

9. The implant of claim 1, wherein the therapeutic agent is selected from the group consisting of therapeutic agents, pharmaceutical agents, biological agents, growth factors, and combinations thereof.

10. The implant of claim 9, wherein the therapeutic agent is selected from the group consisting of antibiotics, analgesics, anesthetics, anti-inflammatory drugs, steroids, anti-viral and anti-retroviral compounds, therapeutic proteins or peptides, therapeutic nucleic acids, and combinations thereof.

11. A spinal implant, comprising:
a load bearing body, having an outer surface, sized for placement at least partially into an intervertebral disc space;
a plurality of sets of reservoirs positioned at least partially within the body;
wherein each set of reservoirs comprises at least one reservoir for holding and releasing therapeutic agents;
and wherein plurality of reservoirs are in fluid communication with at least a portion of the outer surface of the body the body, thereby providing for in vivo release of the therapeutic agents.

12. The implant of claim 11, wherein the body has one or more injection sites for depositing the therapeutic agents.

13. The implant of claim 12, wherein the one or more injection sites include a sealing mechanism to prevent release of the therapeutic agents into the surrounding tissues.

14. The implant of claim 12, wherein the one or more injection sites are marked with at least one x-ray marker.

15. The implant of claim 11, the implant further comprising one or more channels between the at least one reservoir and the outer surface of the body to permit fluid communication therebetween.

16. The implant of claim 15, wherein each set of reservoirs has its own unique set of channels that permit fluid communication between the set of reservoirs and the outer surface.

17. The implant of claim 11, wherein the degree of fluid communication between the reservoirs and the outer surface is different for each set of reservoirs.

18. The implant of claim 11, wherein at least one set of reservoirs is not in fluid communication with any other set of reservoirs.

19. The implant of claim 11, wherein at least one set of reservoirs is in fluid communication with at least one other set of reservoirs.

20. The implant of claim 11, wherein at least one set of reservoirs comprises a plurality of reservoirs, wherein the plurality of reservoirs within that set of reservoirs are in fluid communication with each other.

21. The implant of claim 11, wherein at least one set of reservoirs comprises a plurality of reservoirs, wherein the plurality of reservoirs within that set of reservoirs are not in fluid communication with each other.

22. The implant of claim 11, wherein the body is comprised of one or more materials providing load bearing properties and release of therapeutic agents.

23. The implant of claim 11, wherein the therapeutic agent is selected from the group consisting of therapeutic agents, pharmaceutical agents, biological agents, growth factors, and combinations thereof.

24. The implant of claim 23, wherein the therapeutic agent is selected from the group consisting of antibiotics, analgesics, anesthetics, anti-inflammatory drugs, steroids, anti-viral and anti-retroviral compounds, therapeutic proteins or peptides, therapeutic nucleic acids, and combinations thereof.

25. A method for inserting therapeutic agents into a spinal implant, comprising:
inserting a hypodermic needle into-a spinal implant that includes at least one reservoir positioned at least partially within the spinal implant;
providing an agent through the needle and into the at least one reservoir, the agent being in the form of a liquid solution or suspension;
filling the reservoir at least partially with the agent; and
removing the needle.

26. The method of claim 25, wherein the hypodermic needle is inserted through a predetermined injection site.

27. The method of claim 26, wherein the predetermined injection site includes at least one x-ray marker, and the hypodermic needle is inserted under fluoroscopic guidance.

28. A method for inserting therapeutic agents into a spinal implant, comprising:
creating a pellet containing at least one therapeutic agent in substantially solid; and
fabricating the spinal implant around the pellet so as to embed the pellet at least partially within the spinal implant.

29. The method of claim 28, wherein the pellet is water soluble.

30. A spinal implant prepared by the method of claim 28, wherein the spinal implant has an outer surface and the pellet is in fluid communication with the outer surface of the implant.

31. The method of claim 28, wherein creating the pellet comprises mixing the at least one therapeutic agent with a binder.
